(19) United States
(12) Patent Application Publication
(10) Pub. No.: US 2017/0296586 A1
(43) Pub. Date: Oct. 19, 2017

(54) PHARMACEUTICAL COMPOSITION FOR TREATING CEREBRAL ATROPHY ASSOCIATED DISEASE

(71) Applicant: Gwo Xi Stem Cell Applied Technology Co., Ltd., Zhubei City (TW)

(72) Inventors: Sheng-Tzung TSAI, Zhubei City (TW); Ming-Hsi CHUANG, Zhubei City (TW); Po-Cheng LIN, Zhubei City (TW); Pi-Chun HUANG, Zhubei City (TW)

(21) Appl. No.: 15/478,698

(22) Filed: Apr. 4, 2017

Related U.S. Application Data
(60) Provisional application No. 62/321,946, filed on Apr. 13, 2016.

(51) Int. Cl. A61K 35/28

(52) U.S. Cl. CPC A61K 35/28

(57) ABSTRACT

The present invention provides a pharmaceutical composition to treat cerebral atrophy associated disease. Said pharmaceutical composition comprising adipose-derived stem cells, can reverse the function of excitatory synapses in dementia patients and improve the function of memory storage area in brain.

### CROSS-REFERENCE TO RELATED APPLICATIONS

This Non-provisional application claims priority under 35 U.S.C. §119(a) on Patent Application No(s). [62/321,946] filed in American United States Apr. 13, 2016, the entire contents of which are hereby incorporated by reference.

### FIELD OF THE INVENTION

The present invention relates to a pharmaceutical composition for treating cerebral atrophy associated disease.

### BACKGROUND OF THE INVENTION

Alzheimer's Disease, also known as senile dementia disease, has become a global epidemic as the aging population increases rapidly. According to the International Association dementia statistics, there are 46.8 million global dementia patients in 2015, and one person suffering from dementia disease every 3 seconds. It is expected the global dementia patients will be twice in 2030, thus, how to prevent and treat dementia disease is an important issue.

Dementia disease is a chronic, progressive degenerative disease, can be divided into two categories: degenerative dementia and vascular dementia disease. The majority of patients are degenerative dementia, wherein the Alzheimer's disease, frontotemporal lobe degeneration, and Dementia with Lewy Bodies are the most common disease.

Vascular dementia disease is caused by stroke or cerebral vascular disease, the mental retardation is also caused by brain cell death due to poor blood circulation in the brain. Symptoms of early dementia may include: irritability, aggression, inability to normal speech, easy to get lost, emotional instability, loss of motivation to survive, loss of long-term memory, difficult to remember the recent occurrence, difficult to take care of themselves and behavioral abnormalities. Therefore, when the patient's condition deteriorates, they often begin to leave the family and social relations, and the gradual loss of physical function, then leading to death.

In the current study of dementia prevention is mainly focus on Alzheimer's disease. Although the true cause of Alzheimer's disease is still unknown, the abnormal accumulation of beta-amyloid, neurofibrillary tangles, and extensive neuro-inflammation, are the main pathological features, and so as cause serious impact on nerve repair and regeneration function.

The current treatment of dementia, mainly using acetylcholine inhibitor (acetyl-cholinesterase inhibitor) or NMDA receptor antagonist (NMDA anatagonist) to delay the development of the disease or improve the patient's mental and behavioral symptoms. However, the drugs cannot effectively prevent or restore damaged brain cells, in addition, the above-mentioned drugs on the patients themselves have convulsions, dyspnea and other side effects. Therefore, there is still a need for better medical treatment to treat the dementia disease.

### SUMMARY OF THE INVENTION

Accordingly, the present invention provides a pharmaceutical composition for treating cerebral atrophy associated disease. Said pharmaceutical composition comprising adipose-derived stem cells, can improve the function of excitatory synapses in dementia patients and improve the function of memory storage area in brain.

The present invention provides a method for treating cerebral atrophy associated disease in a subject, wherein the method comprising administering to said subject a pharmaceutical composition, wherein the pharmaceutical composition is having an adipose-derived stem cell.

In one embodiment, the treating cerebral atrophy associated disease is through improving the function of memory storage area in brain of dementia patients.

In one embodiment, the treating cerebral atrophy associated disease is through increasing the function of excitatory synapses in dementia patients.

In one embodiment, the treating cerebral atrophy associated disease is through increasing the numbers of pyramidal neuron synapses in the cerebral prefrontal cortex in dementia patients.

In one embodiment, the treating cerebral atrophy associated disease is through increasing the numbers of pyramidal neuron synapses in the hippocampus in dementia patients.

In one embodiment, the concentration of the adipose-derived stem cells is 6×10⁴/ul˜7×10⁴/ul.

In one embodiment, the pharmaceutical composition is implanted near the fornix area in right cerebral hemisphere, wherein AP is −0.2, ML is −0.5 and DV is −6.

### BRIEF DESCRIPTION OF THE DRAWINGS

FIG. 1A shows the histochemistry stain of brain slices in control group and dementia group, in comparison with control group the histochemistry stain is darker in dementia group.

FIG. 1B shows the amyloid β-protein expressions of control group and dementia group, in comparison with control group the amyloid β-protein expression is higher in dementia group.

FIG. 2A shows the histochemistry stain of brain slices in the experimental group in day 3, wherein the blue fluorescence is DAPI and the yellow fluorescence is Anti-Human nuclei antigen (MAB1281).

FIG. 2B shows the histochemistry stain of day 14 brain slices in the experimental group, wherein the blue fluorescence is DAPI and the yellow fluorescence is Anti-Human nuclei antigen (MAB1281)

FIG. 3 shows the spatial memory learning analysis of rats in all group, wherein the points in the figures represent the escaping time from water maze, the longer time represents the worse memory function; wherein the memory function of rats in the experimental group is improved as similar as rats in the control group, and is significantly different with the rats in dementia group (the rats in dementia group are having worse memory function) in day 5.

FIG. 4 shows the test result of excitatory synapses function of rats in all groups; as shown in the figure, the glutamatergic postsynaptic expression of rats in dementia group is significantly lower than the rats in control group; however, the glutamatergic postsynaptic expression of rats in experimental group is significantly higher than the rats in dementia group.

FIG. 5A shows four portions of medial prefrontal cortical pyramidal neurons of rats, which includes: proximal end of basal dendrite, distal end of basal dendrite, proximal end of apical dendrite and distal end of apical dendrite, calculating the numbers of neurons, wherein the proximal end means closer to neuron body.

FIG. 5B shows the statistic data of dendritic spine density of medial prefrontal cortical pyramidal neurons of rats, in comparison with control group, the numbers of dendritic spine in dementia group is decreased; however, the dendritic spine density of degenerated neurons in experimental group is increased significantly, which is similar to the dendritic spine density of degenerated neurons in control group

FIG. 6A shows the pictures of all portions of CA1 hippocampal pyramidal neurons, which is photographed by 10 μm from 2nd branch of distal end of basal dendrite near neuron body, and from the 3rd branch of apical dendrite near neuron body.

FIG. 6B shows the statistic data of dendritic spine density of CA1 hippocampal pyramidal neurons, wherein the in comparison with the control group, the numbers of dendritic spine in dementia group is decreased; however, the dendritic spine density of degenerated neurons in experimental group is increased significantly, which is similar to the dendritic spine density of degenerated neurons in control group

### DETAILED DESCRIPTION OF THE INVENTION

Adipose-derived stein cells (ADSCs) of the present invention are mesenchymal stem cells (MSC) in adipose tissue, which can be obtained from liposuction or lipectomy. That means, it is easy and convenient to obtain the adipose-derived stem cells without causing trauma in patients. Besides, the adipose-derived stein cells can be autologous implanted, which is easily be compatible, so as to reduce the anti-rejection drugs, long-term in vitro culture and other shortcomings. Furthermore, the adipose-derived stem cells are easy to isolate and can be rapidly and stably amplified in vitro, and are also less susceptible to aging, thus being suitable for use as pharmaceutical compositions of the present invention.

Experimental Animals

The Wistar rats are divided into three groups: control group, dementia group and experimental group, 300 pmole Amyloid-β-protein is implanted daily into the rats in dementia group and experimental group in day 0-28, the implantment position is AP: 0.4, ML: 1.5, DV: 3.3 so as to cause dementia disease. The pharmaceutical composition of the present invention (containing 1×10⁶/15 ul adipose-derived stem cells, which is 6×10⁴/ul˜7×10⁴/ul adipose-derived stem cells) is implanted into the rat brain of experimental group in day 14, the implantment position is near the fornix area in right cerebral hemisphere (AP: −0.2, ML: −0.5, DV: −6) and the rat brain in control group is punctures in day 14.

### Example 1. Amyloid β-Protein Staining

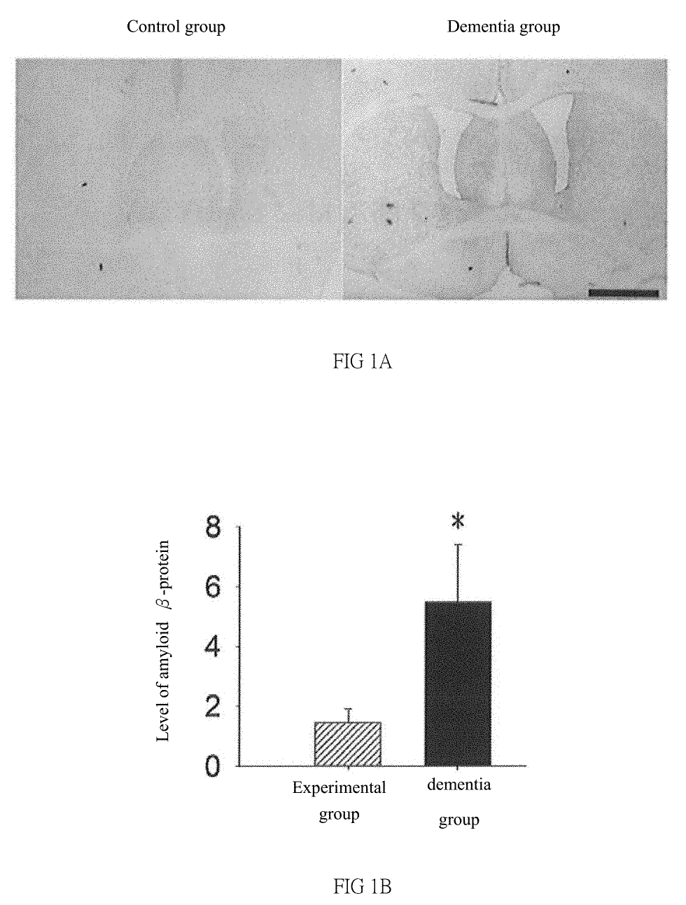

The rat brain of control group and dementia group is stained by anti-Aβ 1-42 antibody. Please refer to FIG. 1A-1B, the historical staining color of dementia group is darker than control group, therefore, the level of amyloid β-protein of dementia group is higher.

### Example 2. Immunofluorescence Staining

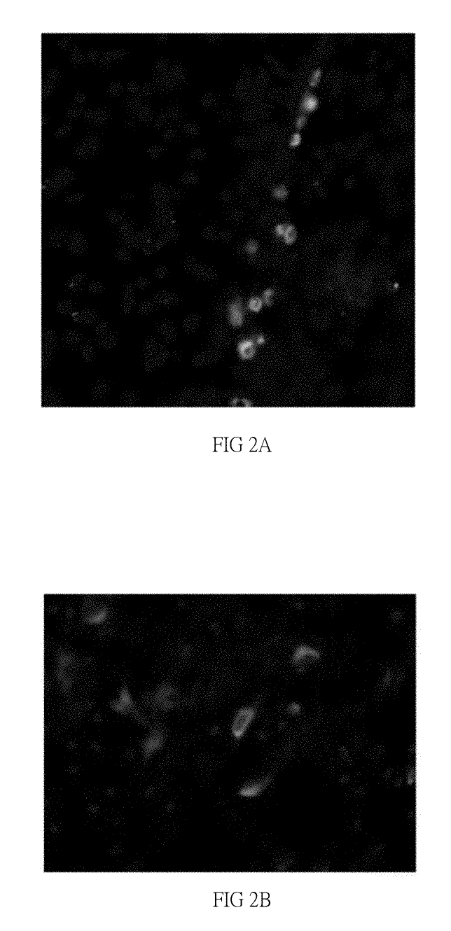

The rats of experimental group is implanted with the pharmaceutical composition of the present invention, and then sacrificed in day 3 (FIG. 2A) and week 2 (FIG. 2B) respectively. The brain samples are stained by anti-Human nuclei antigen (MAB1281), the results are shown in FIG. 2A and FIG. 2B, wherein the blue fluorescence is DAPI and the yellow fluorescence is Anti-Human nuclei antigen (MAB1281).

In FIG. 2B, the Adipose-derived stem cells are survive in rat brain after the pharmaceutical composition implantment for 14 days, wherein the cell morphology is same as the cells in rat brain after the implantment for 3 days (FIG. 2A).

### Example 3. Spatial Memory Test

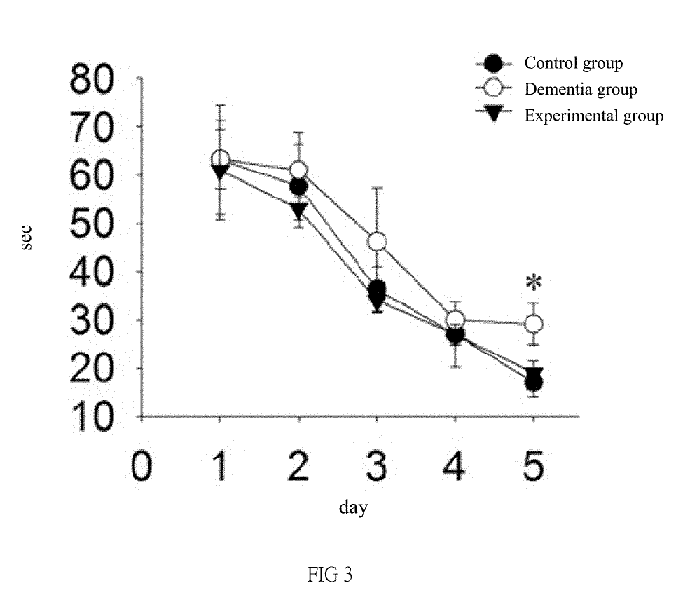

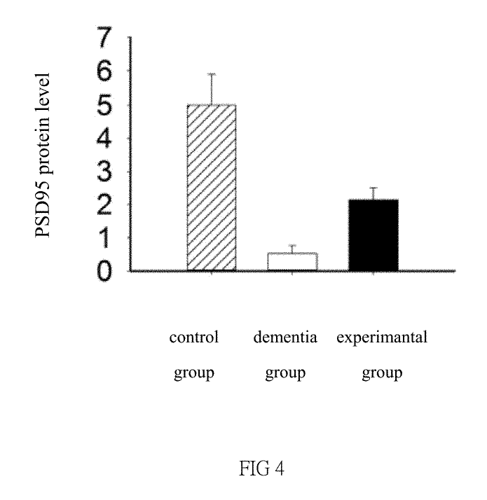

The rats in control group, dementia group and experimental group were placed in a Morris water maze for 5 days to observe the movement patterns of rats in each group for the test of spatial memory. Please refer to FIG. 3, the points represent the various groups of rats to escape the water maze of time, the longer time means the worse memory function. The results of the day 5 in the experimental group showed that the memory function of the experimental group was improved to be similar to the control group (50% to 60% improvement in the experimental group compared with the dementia group) and significantly different from the dementia group (dementia group are having worse memory function), it can be seen that the pharmaceutical composition of the present invention can improve the function of memory storage area in brain of dementia patients.

### Example 4. Efficacy Test of Excitatory Synapses

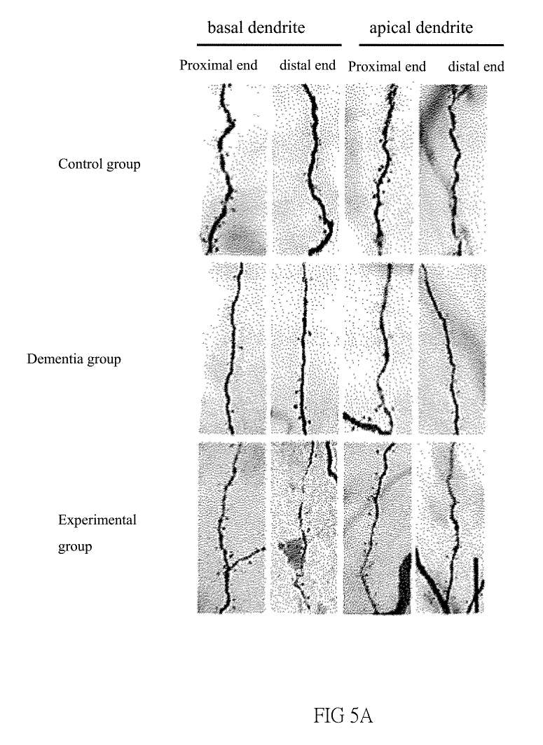

The rat brain of control group, dementia group and experimental group rats were analyzed by Western-style dot-blot method using the antibody of synaptic density protein 95 (PSD-95). The level of glutamatergic postsynaptic in the brain of the rat is shown in FIG. 4.

As shown in FIG. 4, the level of glutamatergic postsynaptic in the dementia group was significantly decreased (80% to 90% in the dementia group compared with the control group). However, the level of rat brain glutamate acid synaptic (glutamatergic postsynaptic) in experimental group is higher than dementia group (50% to 60% higher in the experimental group compared with the dementia group). Therefore, the pharmaceutical composition of the present invention is effective to improve the function of excitatory synapses in dementia patients.

### Example 5. Dendritic Spine Density Analysis

The neurons in the control group, dementia group and experimental group rats were labeled with Lucifer yellow to analyze the density of nerve dendrites in the brain of each group.

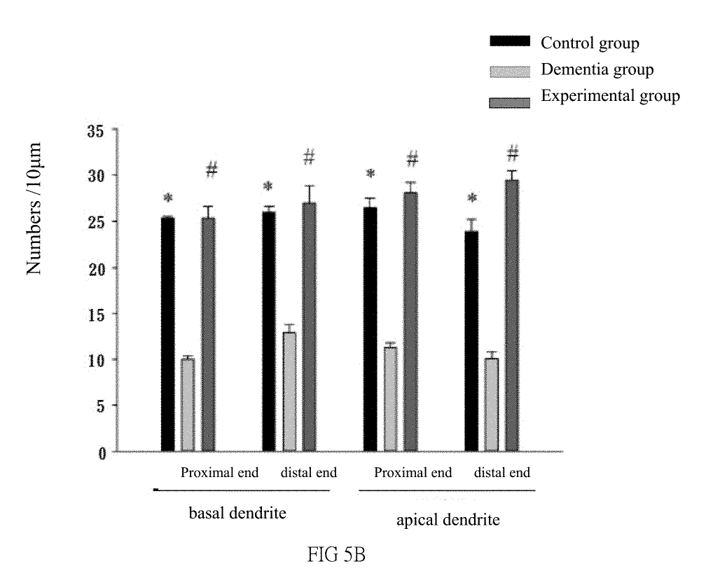

FIG. 5A shows the dendritic spine density of four portions of medial prefrontal cortical pyramidal neurons of rats, which includes: proximal end of basal dendrite, distal end of basal dendrite, proximal end of apical dendrite and distal end of apical dendrite, calculating the numbers of neurons, wherein the proximal end means closer to neuron body. The calculating method is to count the number of synapses, and then calculate the mean value within a distance of 10 μm in four representative regions of each group in the pictures. Statistical results as shown in FIG. 5B, the rats in dementia group had significantly lower nerve synapses compared with the control group (40% to 45% less in the dementia group than in the control group), whereas the experimental group showed the number of synapses in degenerative nerves increased significantly (the experimental group increased by 90% to 108% compared with the dementia group), and the number of synapses with the control group recovered to similar amount with control group. Therefore, the pharmaceutical composition of the present invention is effective to increasing the numbers of pyramidal neuron synapses in the cerebral prefrontal cortex in dementia patients.

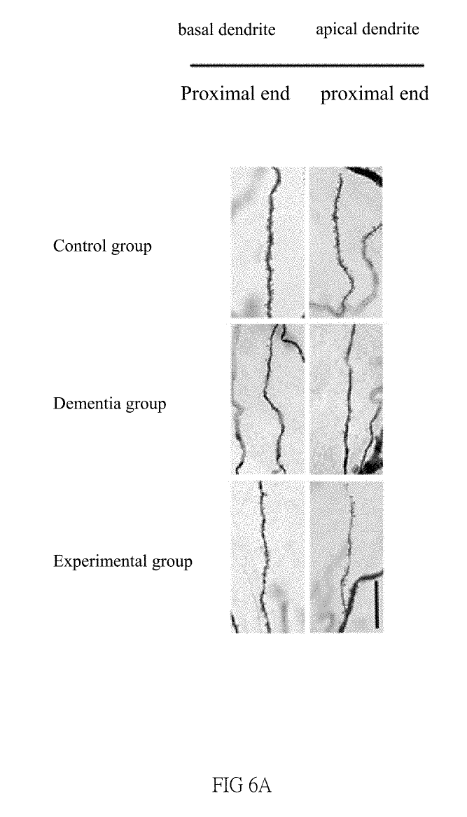

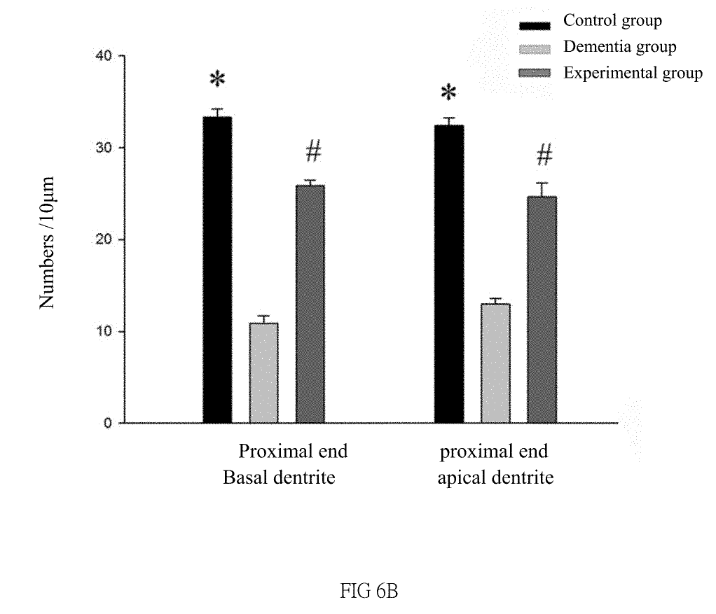

FIG. 6A shows the pictures of all portions of CA1 hippocampal pyramidal neurons, which is photographed by 10 μm from 2nd branch of distal end of basal dendrite near neuron body, and from the 3rd branch of apical dendrite near neuron body. The number of synapses in the picture is counted, and then average was calculated.

Statistical results is shown in FIG. 6B, the dementia group had significantly lower neuronal synapses compared to the control group (30% to 40% reduction in the dementia group compared with the control group), whereas the experimental group showed degenerative nerves processes (Experimental group increased 70%˜80% compared with dementia group), and number of synapses is recovered to similar amount with control group, it can be seen that the pharmaceutical composition of the present invention can increase the numbers of pyramidal neuron synapses in the hippocampus in dementia patients.

From the above results, the pharmaceutical composition of the present invention can effectively increase the density of nerve dendrites of dementia patients.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method for treating cerebral atrophy associated disease in a subject, wherein the method comprising administering to said subject a pharmaceutical composition; wherein the pharmaceutical composition is having an adipose-derived stem cell.

2. The method of claim 1, wherein the treating cerebral atrophy associated disease is through improving the function of memory storage area in brain of dementia patients.

3. The method of claim 1, wherein the treating cerebral atrophy associated disease is through improving the function of excitatory synapses in dementia patients.

4. The method of claim 1, wherein the treating cerebral atrophy associated disease is through increasing the numbers of pyramidal neuron synapses in the cerebral prefrontal cortex in dementia patients.

5. The method of claim 1, wherein the treating cerebral atrophy associated disease is through increasing the numbers of pyramidal neuron synapses in the hippocampus in dementia patients.

6. The method of claim 1, wherein the concentration of the adipose-derived stem cells is 6×10⁴/ul˜7×10⁴/ul.

7. The method of claim 1, wherein the pharmaceutical composition is implanted near the fornix area in right cerebral hemisphere, wherein AP is −0.2, ML is −0.5 and DV is −6.
